(19) United States
(12) Patent Application Publication
(10) Pub. No.: US 2017/0188900 A1
(43) Pub. Date: Jul. 6, 2017

(54) MEMS SPECTROMETER 2-DISK SYSTEM

(71) Applicant: Steven C. White, Las Vegas, NV (US)

(72) Inventor: Steven C. White, Las Vegas, NV (US)

(21) Appl. No.: 15/069,933

(22) Filed: Mar. 14, 2016

Related U.S. Application Data
(60) Provisional application No. 62/133,223, filed on Mar. 13, 2015.

(51) Int. Cl. A61B 5/1455; A61B 5/145; A61B 5/00; G01J 3/02

(52) U.S. Cl. CPC A61B 5/1455; G01J 3/0202; G01J 3/0272; A61B 5/14546; A61B 5/0002

(57) ABSTRACT

A system for detection of blood trace analytes is disclosed. The system comprises an illumination disc supporting a plurality of sources of electromagnetic radiation and rotatable relative to an interdigital location of a subject, and a spectrometer disc supporting a plurality of spectrometers able to process the electromagnetic radiation from the illumination disc and rotatable relative to the interdigital location of a subject at a position opposite that of the illumination disc.

### CROSS-REFERENCE TO RELATED APPLICATION(S)

This application claims the benefit and the priority of U.S. Provisional Patent Application No. 62/133,223 filed Mar. 13, 2015, the entire contents of which are incorporated herein by reference.

### SUMMARY

This invention relates to the field of medical devices for non-invasive blood trace analyte detection, measurement, and medical analysis thereof. More specifically, one or more embodiments of the invention comprise non-invasive measurement system with a MEMS spectrometer disc, and an illumination disc (2disc system) that rotate, controlled by a micro positioning control panel, to measure all blood constituents with absorption between 100 nm-5000 nm. The light source assigned to a specific wavelength region coupled with the MEMS spectrometer chip specific to that same wavelength region will use a transmission spectroscopy method.

One or more embodiments of the system will work by using a microprocessor to position MEMS spectrometers (rotational position on the y-axis), in position with a light source from an illumination disk (rotational position on the x-axis) to ensure alignment based on wavelength. The coordinated positioning coordinates the electromagnetic radiation emission and the MEMS spectrometers that correspond to processing a specified electromagnetic radiation wavelength region. This coordinated system will allow for non-invasive detection and quantification of multiple analytes detectable from wavelengths from 100 nm-5000 nm wavelengths.

The form of spectroscopy covers analysis of any blood trace analyte whereby the illumination disc, consisting of several different light sources, located on bottom (palmar aspect) side, emits electromagnetic radiation to pass through the arterial blood supply (palmar artery), and the MEMS spectrometer disc (dorsal aspect), consisting of several MEMS spectrometers, receives the light information that has passed through the interdigital spacing between illumination disk and the MEMS spectrometer disk. This may use several MEMS chip spectrometers that cover certain wavelength regions, and several light sources from the illumination disc that correspond to the wavelength regions of interest. See FIG. 1.

The method for non-invasively evaluating blood trace analytes of a subject may include providing a source of electromagnetic radiation to a device, positioning the MEMS chip spectrometer disc at an interdigital location between fingers or toes of a subject, and receiving the electromagnetic radiation that has passed through or reflected from the subject between the interdigital space between the proximal phalanx and between the adjacent metacarpophalangeal joints. See FIG. 6.

In an aspect of an embodiment of the present invention, the method includes applying the device to a surface of the subject at a substantially constant pressure, and the device can be applied such that it is it to be substantially flush with the surface of the subject's skin. The device may also include a touch and/or other type of sensor to activate the device and/or a pressure sensor to ensure the device is operated at a particular pressure. However, it should be understood that the device may also be applied so as to be spaced from the surface of the subject's skin.

In an aspect of embodiment of the present invention, the system includes a MEMS spectrometer disc (positioned at the dorsal aspect between the metacarpophalangeal joint) that rotates along the y-axis plane, allowing for non-invasive measurement of blood analytes detectable from 100 nm-5000 nm through a series of MEMS spectrometers, and the illumination disc (positioned at the palmar aspect between the metacarpophalangeal joint) with several lights sources that rotate along the x-axis palmar plane to provide electromagnetic radiation from 100 nm-5000 nm.

In an aspect of an embodiment of the present invention, the electromagnetic radiation can be near infrared radiation, fluoroscopy, pulse oximetry, and include diffuse reflectance or transmission spectroscopy methods.

In another aspect of an embodiment to the present invention, an apparatus is provided for non-invasively evaluating blood trace analytes of a subject including a means for providing a multiple sources of electromagnetic radiation to a device, a means for positioning the device at an interdigital location between fingers or toes of the subject, and a means for receiving multiple electromagnetic radiation signals absorbed and or reflected from the subject. Receiving the electromagnetic radiation may occur on the dorsal aspect of the interdigital space, whereby the illumination occurs from the palmar aspect. See FIG. 1.

Another aspect of an embodiment of the present invention is directed toward a multiple spectrometer plus multiple light source detection system for non-invasive evaluation of body fluids of a subject. The system includes multiple sources of electromagnetic radiation and may or may not use probe light source(s), light pipe(s) or fiber optic(s). Conveyance of the electromagnetic radiation from the illumination disk to the MEMS Spectrometer Disk may or may not include fiber optics. The integration of choosing light source to MEMS chip corresponds to the wavelength region of interest whereby the emitted electromagnetic radiation at specific wavelength regions is in parity with the illumination lights source from disk. The system can operate at any interdigital location between fingers or toes of the subject. The illumination disk may consist of multiple light sources that transmit light from the palmar aspect between two metacarpophalangeal joints, and the MEMS spectrometer detectors will receive information from the dorsal aspect between two metacarpophalangeal joints. See FIG. 6 Target Measurement Zone and FIG. 2.

In another aspect of an embodiment of the present invention, the MEMS spectrometer disk and illumination disk detection light system includes a source of electromagnetic radiation covering wavelength regions from 100 nm-5000 nm capable of transmission spectroscopy.

### BRIEF DESCRIPTION OF THE DRAWINGS

FIG. 1 is a schematic side view of a system according to the present invention.

FIG. 2 is a schematic side view of another system according to the present invention.

FIG. 3 is a schematic top view of a component that could be used in a system according to the present invention.

FIG. 4 is a schematic top view of a user interfacing with a component that could be used in a system according to the present invention.

FIG. 5 is a schematic bottom view of the component of FIG. 4.

FIG. 6 is a diagram of a circulatory system in a patient's hand showing potential target zones for use of the present invention.

### DETAILED DESCRIPTION

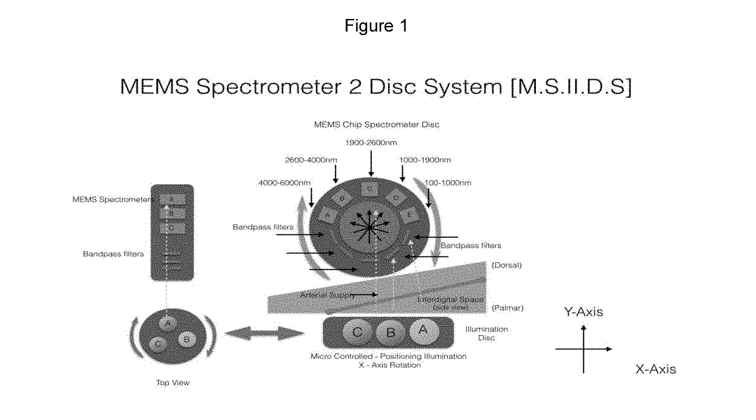

FIG. 1 illustrates a side view of a system interface having a MEMS spectrometer disk with multiple spectrometers and detector, which may or may not be directly attached to the MEMS spectrometer. The system may or may not include fiber optics. The illumination disk (light source) may or may not include fiber optics, and a mechanism connecting the MEMS spectrometer disk to the illumination disk. The connection may include any material or bracket that allows the illumination disk and MEMS spectrometer disks to ensure flush positioning with the interdigital space. The light source(s) will emit from the palmar aspect with detection/spectrometer(s) on the dorsal aspect positioned between any interdigital space of the hand, and or the interdigital space of the feet at the palmar digital crease. The MEMS spectrometer disk and illumination disk may be flush with skin and are ergonomically positioned using the system interface. The method of spectroscopy covers any form of electromagnetic radiation at any wavelength and any type of detector whereby the light source is positioned with respect to the appropriate detecting spectrometer.

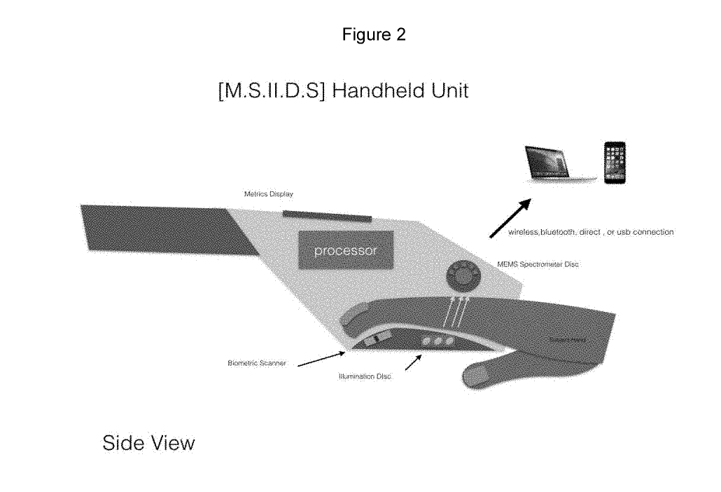

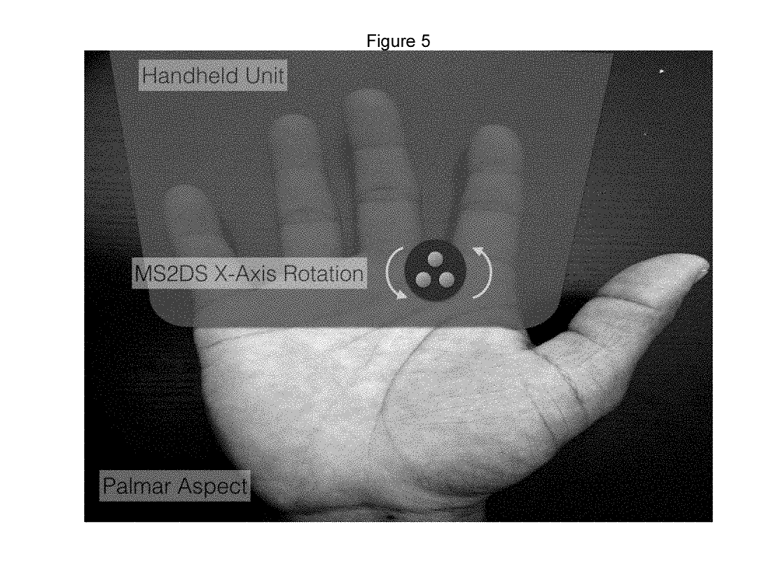

FIG. 2 illustrates a method of spectroscopy integrated into a handheld unit where the system is integrated into the handheld unit. A processor that ensures proper alignment of illumination disk and spectrometer disk will control the system compact XY axis interface. The system handheld unit may also include several ergonomic enhancements such as palmar padding. Light source(s) emits from the palmar aspect, and detection occurs at the dorsal aspect. Measurement site occurs at the interdigital space between the first (index) finger and the second (middle) finger, but is not limited to this region and can include any interdigital space whereby arterial blood supply is targeted for detection, measurement, and or analysis of measureable blood trace analytes. See FIG. 5. The system also illustrates the ability to communicate subject information to a mobile device, cell phone, computer, patient database, or computer software. Biometric identification capabilities are also possible to exclusively identify patient, and ensure HIPAA compliance.

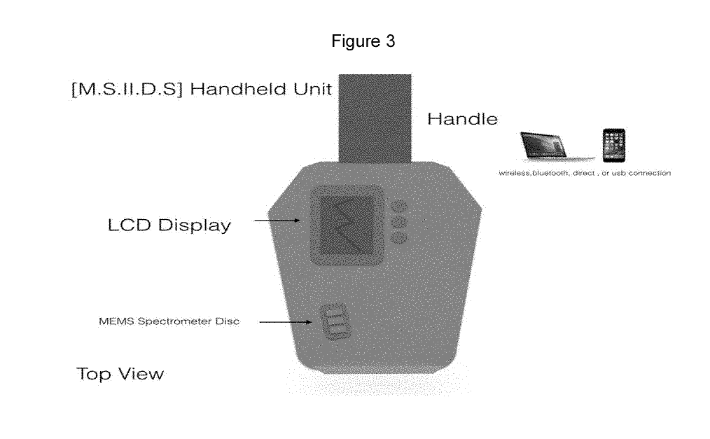

FIG. 3 illustrates the top view of a system handheld unit. System location: Between the knuckles of any interdigital spacing whereby the system targets arterial blood supply for detection, measurement, and medical analysis of blood analytes measured at this site. It also illustrates the location and method of spectroscopy whereby the MEMS spectrometers collect from the dorsal aspect. The illustration is an artistic rendering of a system handheld unit to illustrate top view and where a subject inserts a hand for measurement.

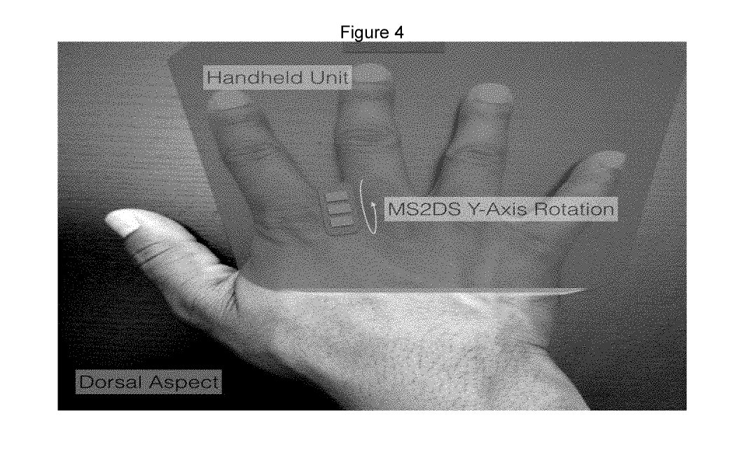

FIG. 4 illustrates the top view of the hand (dorsal) integrated into the system handheld unit. System measurement location: Between the metacarpophylangial joints of any interdigital spacing whereby the system collects electromagnetic radiation directly through arterial blood supply for detection, measurement, and medical analysis of blood analytes at this site. It also illustrates the location of spectroscopy whereby the detection source is located on the dorsal aspect. The illustration is an artistic rendering of what the system will look like from the dorsal aspect. The MEMs spectrometer source(s) disc may or may not include a fiber bundle(s) or light pipe(s).

FIG. 5 illustrates the bottom view of the hand (Palmar) integrated into the system handheld unit. The system measurement location: between the palmar beds on the palmar aspect of the metacarpophalangeal joint.

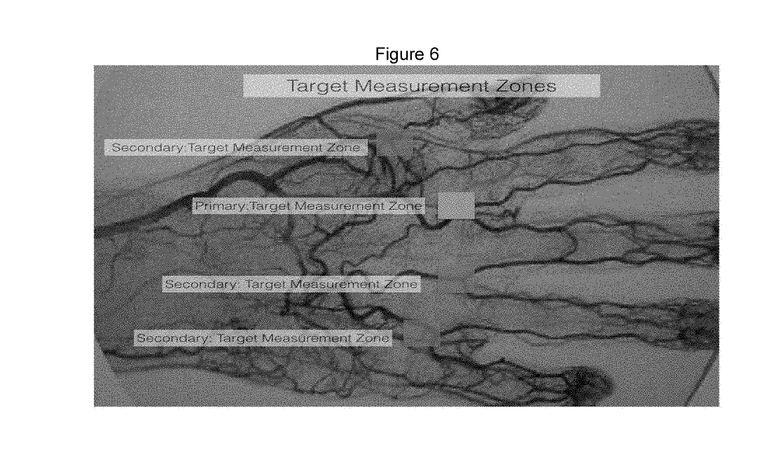

FIG. 6 illustrates the target measurement zones “TMZs” (Arterial Blood Supply). The TMZ is the arterial blood supply from the digital palmar artery. The TMZ is at the interdigital space between the metacarpophalangeal joints.

An apparatus for non-invasive spectroscopic measurement according to the one embodiment of the present invention is suitable for many applications, particularly for non-invasively evaluating blood trace analytes such as: minerals, organic, and inorganic compounds, pharmaceutical drugs, synthetic markers or nano-particles. In addition, the apparatus could be used to detect toxins or hazardous chemicals in the blood.

Such an apparatus could be useful in biomedical applications. For example the apparatus could be used as a screening device to quickly analyze blood. In such applications, the apparatus could be configured to be attachable to a patient for continuous monitoring whereby the system is integrated into a hand held unit. Continuous monitoring would allow for real time monitoring of blood samples to track fluctuations of blood analytes. When coupled with a biometric identification device, the apparatus could also link to insurance and/or medical records of a patient. This link could be used to update patient blood panel information or to make comparisons of past blood panel readings; serving as a valuable instrument in monitoring and tracking continuous changes patient blood over time.

Such an apparatus could be useful in the biomedical application of complete blood analysis device. The system may be capable of measuring and detecting all blood constituents with absorption between 100 nm-5000 nm. This technology can transmit via Bluetooth, or direct sync to mobile device, cell phone, or computer. The valuable data can then be stored in a database whereby international blood profiles can be compared, analyzed, or referenced.

Multiple embodiments include many different light sources within the illumination disk to provide multiple sources of electromagnetic radiation within the 100 nm-5000 nm windows. In such an embodiment, for example, a quartz halogen lamp is used to provide a source of electromagnetic radiation in the near infrared region; suitable for non-invasive measurement of concentrations of certain blood components or blood analytes, such as alcohol or glucose. Other light producing devices such as flash lamps, tungsten-halogen, lights, light emitting diodes, quartz halogen, or laser sources can be used in conjunction with filtering mechanisms to produce a certain spectral range that corresponds to the spectral range absorption of other targeted tissue components or analytes to be measured. The capability of transmission of multiple forms of electromagnetic radiation from the palmar aspect at the interdigital space between the metacarpophalangeal (MCP) joint and multiple receiving MEMS spectrometer & detectors on the dorsal aspect of the interdigital space between the metacarpophalangeal (MCP) joint is part of what makes this invention unique. Additionally, the system will integrate into a handheld device that is easily transportable, and ergonomically designed to ensure proper fit, placement, and ergonomic comfort of subject hand will in the system handheld unit. The XY multi-axis multi axis illumination disk and MEMS spectrometer disc system is an additional design component that ensures multiple measurement capabilities across a 5000 nm region. The system may/may not use fiber optics or light pipes. The system may/may not be connected to LCD screens, which will display/process measurement metrics/information, which can transmit signal via blue tooth or direct connection to a computer, wireless device, or processing software system.

MEMS spectrometer disk plus illumination disc will consist of a light source disk, “illumination disk,” and a MEMS spectrometer/detector disk. Both disks will operate in perpendicular form along an X and Y axis system. The XY rotational sync system will ensure proper alignment of the light source with MEMs detector; ensuring proper wavelengths of electromagnetic radiation corresponding to the proper spectrometer. The illumination disk rotates on the X-axis, and the MEMS spectrometer disk rotates on the Y-axis.

Spectrometer/detector: May or may not include fiber bundle(s) or light pipe(s). Detector/sensor may or may not be a probe, collection fiber bundle, or chip sensor. Detector may or may not be directly attached to the spectrometer.

Light: May or may not include fiber bundle. Light source can be any electromagnetic radiation: a quartz halogen, laser, light emitting diode, or a probe at the tip of light source fiber optics. Light source may integrate several light source types in a disk orientation that will rotate for optimal emission corresponding to targeted blood analyte.

Interdigital tissue interface: The interface may consist of any malleable material that allows consistent pressure of the detector and light source to position flush with the interdigital space between the hand or feet digits. The interdigital tissue interface can be integrated into a handheld device, hand cradle, adjustable clip, ergonomic bracket, etc.

Two disk connection system: The system may consist of a bracket that allows for proper alignment of the illumination disk and MEMS spectrometer disk, and allow for adjustment to properly adjust to difference hand shaped and sized. The illumination disk may be stationary, as it may be placed on the bottom of the handheld unit, whereby the movement of the MEMS spectrometer disk will move up/down along the Y-axis.

Material: Can be any resilient material with memory, and may include but not limited to: plastic, aluminum, carbon, rubber, latex, fabric, neoprene, etc.

The system can couple with biometric identification capabilities to share information with handheld devices, mobile phones, computers, patient databases or any software integrated into the aforementioned devices capable of receiving such.

An apparatus for spectroscopic evaluation of a subject's body fluids may be used at the interdigital region adjacent to or in between a subject's extremities using spectroscopy. The spectroscopy positioning consists of multiple MEMS spectrometers/detectors on a disc at the dorsal aspect, and multiple light sources on a disc on the palmar aspect. The spectroscopic measurement system may be positioned flush with or spaced from skin at both palmar/dorsal aspects of hand measuring between the second and third proximal phalanx and between the (MCP) metacarpophalangeal joint. The imaging system which may include spectroscopy or fluoroscopy, targets the palmar digital arterial blood supply at the interdigital spacing between any hand or foot digit (see FIG. 6) and can detect and quantify multiple blood analytes using spectroscopy methods. The spectroscopy system can be used to measure multiple blood trace analytes such as: blood alcohol, blood glucose, pharmaceuticals, drugs, blood oxygenation levels, sodium, magnesium, urea, calcium, chloride, carbon dioxide, creatinine, potassium, lactic acid and or pulse/heart rate for any medical screening or diagnostic purposes to analyze a blood panel. The spectroscopic device may possess one or more of the movements such as rotational, translational, and/or vertical freedom necessary for the disks or interface to contact the subject's tissue at a consistent angle and pressure while accommodating the different size of the subjects extremities, and may be of any memory yielding material optimized for attaining reproducible blood flow to the region of the subject that is measured, and for minimizing the effects of pulling, stretching, pressing, compressing the subject's skin. In addition, the spectroscopic measurement device may be coupled with a temperature measurement means that detects the subject's body temperature in or near the region being measured, or the subject's core or mean body temperature, or the ambient temperature proximate to the probes, detectors, or sensors. In addition, the spectroscopic measurement may be coupled with a biometric scanner that detects the subject's identity. The biometric scanner can be positioned to measure the fingerprint of any digit on the hand or foot. The system may also include one or more methods to communicate information of a subject to a mobile device, computer, cell phone, subject database, or computer software.

What is claimed is:

1. A system for detection of blood trace analytes comprising:
an illumination disc supporting a plurality of sources of electromagnetic radiation and rotatable relative to an interdigital location of a subject;
a spectrometer disc supporting a plurality of spectrometers able to process the electromagnetic radiation from the illumination disc and rotatable relative to the interdigital location of a subject at a position opposite that of the illumination disc.

2. A system according to claim 1 wherein the sources of electromagnetic radiation cover wavelength regions from 100 nm to 5000 nm.

3. A system according to claim 1 wherein the illumination disc and the spectrometer disc are positioned relative to the subject to allow for transmission spectroscopy.
